(19) United States
(12) Patent Application Publication
(10) Pub. No.: US 2018/0207204 A1
(43) Pub. Date: Jul. 26, 2018

(54) KRILL OIL PREPARATIONS WITH OPTIMAL MINERAL AND METAL COMPOSITION, LOW IMPURITIES AND LOW AND STABLE TMA LEVELS

(71) Applicant: AKER BIOMARINE ANTARCTIC AS, STAMSUND (NO)

(72) Inventors: Liat Ronen, Zichron Yaakov (IL); Ran Numa, Haifa (IL); Gai Ben-Dror, Gita (IL)

(73) Assignees: ENZYMOTEC LTD., Migdal Ha'emeq (IL); ENZYMOTEC LTD., Migdal Ha'emeq (IL)

(21) Appl. No.: 15/118,296

(22) PCT Filed: Feb. 10, 2015
(86) PCT No.: PCT/IB2016/000131

§ 371 Date: Aug. 11, 2016

Related U.S. Application Data
(60) Provisional application No. 61/938,599, filed on Feb. 11, 2014.

(51) Int. Cl. A61K 35/612; A23D 9/007; A23D 9/02; A23L 33/115; A23L 33/00; A61K 31/132; A61K 33/06; A61K 9/48

(52) U.S. Cl. CPC A61K 35/612; A23D 9/007; A23D 9/02; A23L 33/115; A23V 2200/3262; A61K 31/132; A61K 33/06; A61K 9/48; A23V 2002/00; A23L 33/30

(57) ABSTRACT

A Krill oil preparation including a reduced amount of trimethylamine of 5 mgN/100 g or less, or more than 700 ppm by weight of endogenous calcium and/or less than 1200 ppm by weight of sodium. A process of making such a Krill oil preparation that includes a step of extracting Krill oil from Krill with a solvent mixture of one or more polar solvents and one or more non-polar solvents. A method of treating a human in need of treatment that includes administering to the human an amount of such a Krill oil preparation.

The present application claims priority to the U.S. Provisional Patent Application Ser. No. 61/938,599, filed on Feb. 11, 2014, the disclosure of which is herein incorporated by reference in its entirety.

### FIELD OF THE INVENTION

The present invention relates to Krill oil preparations with low and stable amounts of trimethylamine (TMA) and/or optimal mineral and metals content and/or low amounts of impurities.

### BACKGROUND OF THE INVENTION

Epidemiological and clinical studies have shown various health benefits with consumption of fish and sea foods. These positive health outcomes are attributed to the presence of long chain n-3 polyunsaturated fatty acids (LC-PUFA) in foods, including eicosapentaenoic acid (EPA) and docosahexaenoic acid (DHA) (Ruxton et al. 2004). Studies have demonstrated that the bioavailability of n-3 LC-PUFA attached to phospholipids is better than that of n-3 LC-PUFA attached to glycerol (Ramprasath et al. 2014; Schuchardt & Hahn 2013).

Minerals are required for normal physiological processes and are an essential part of a healthy diet. Krill accumulates metals and minerals and therefore may play an important role delivering these nutrients (Tou et al. 2007).

Calcium is an essential macromineral for several physiological functions and is necessary, for example, for the proper formation and maintenance of strong bones and constriction and relaxation of blood vessels. Calcium is also necessary for the proper functioning of enzymes throughout the human body (Power et al. 1999).

Supplementation of pure Krill oil with exogenous (synthetic or from other sources) essential minerals and metals, such as calcium, has several potential disadvantages: (1) Potential formation of unknown complexes between the exogenous ingredients and Krill oil, which may have an impact on the digestion, absorption and biological activity of the active ingredients of the Krill oil; (2) reduced synergism between endogenous minerals or metals and Krill oil; (3) potential decomposition of active ingredients and reduction of shelf life stability; (4) accumulation of undesirable contaminants that may result from the processes by which the exogenous compounds were produced; and (5) increased allergenic potential.

An additional important mineral for the human body is sodium. The human body needs a small amount of sodium in order to maintain a balance of body fluids and for the proper functioning of organs. However, it is well known that excessive sodium intake has negative effects on human health. High levels of sodium in the diet draw water into the blood-stream, increase blood pressure (also known as hypertension), and as a result, the risk of heart disease, kidney disease, and stroke is increased (Aburto et al. 2013). Therefore, the world health organization encourages the consumption of a diet (including food and dietary supplements) that contains only minimal amounts of sodium (World Health Organization: Guideline Sodium intake for adults and children, 2012).

Clinical trials found that Krill oil consumption positively manages blood lipids in hyperlipidemic patients by significantly reducing triglyceride, total cholesterol and LDL-cholesterol levels. At the same time, Krill oil consumption may increase HDL-cholesterol levels and reduce glucose levels in the blood. Hence, Krill oil consumers represent a population that is particular susceptible to the blood pressure-raising effects of sodium. Therefore, the sodium content in Krill oil supplements should be minimized as much as possible.

Another parameter which determines the quality of Krill oil is the amount of trimethylamine (TMA) that is present. TMA is one of the molecules included in total volatile nitrogen (TVN) and produced during the decomposition of dead organisms. Thus, high levels of this compound indicate a lack of freshness of the tested marine raw material. Specifically, a European Union directive on fish hygiene provided instructions according to which inspectors must conduct TVN/TMA-based chemical testing if the organoleptic examination of fish reveals any doubt as to its freshness (Council Directive 91/493/EEC of 22 Jul. 1991; Laying down the health conditions for the production and the placing on the market of fishery products; Official Journal L 268, Sep. 24, 1991, pp. 0015-0034). Thus, the TMA levels of marine raw materials used for food production (e.g., Krill) are often used as an indicator of the materials' spoilage/freshness and the potential need for further treatment of the materials (Baixas-Nogueras S et al.; Trimethylamine and total volatile basic nitrogen determination by flow injection/gas diffusion in Mediterranean hake (Merluccius merluccius); Journal of Agricultural and Food Chemistry April 49(4):1681-1686; 2001).

Lower TMA levels in food are important and beneficial for a number of reasons: The AIHA reported in 2005 that inhalation of TMA may cause respiratory irritation in humans. Another source (Deichmann and Gerarde 1969) reported that “methylamines” inhalation may cause irritation of the nose and throat, larynx constriction, difficulty breathing, and lung edema [ACUTE EXPOSURE GUIDELINE LEVELS (AEGLs) FOR TRIMETHYLAMINE (CAS Reg. No. 75-50-3), INTERIM].

TMA is known to be formed as part of the degradation of non-processed dead marine organisms, when enzymes and bacteria are still active (D. Greed and P. D. Tom, Measuring, Maintaining Freshness in Aquaculture Products, Global Aquaculture Advocate, September/October 2006, p 40-42). It was therefore assumed that once the marine material is processed (usually by heating and drying), and thus contains no enzymatic and bacterial activities any longer, the TMA levels will not increase. Further, it was assumed that once oil is extracted from such dead marine organisms, using solvent extraction, thermal treatment or both, the level of the TMA in the oil will not increase. Therefore, once a low TMA in Krill oil is achieved, the TMA level is expected to remain stable during storage (due to the absence of any enzyme or bacteria activity) and thus no routine tests of the TMA levels over time are conducted with respect to Krill oil. It is now discovered that TMA levels can increase over time when extracted using conventional extracted methods. Therefore, there is a need to provide a method of extraction that achieves a low TMA level that remains stable during storage.

It is also desirable that Krill oils have low impurity levels (such as choline, betaine, and amino acids), high internal antioxidant capacity and optimal lipase activity. Omega-3 rich oils such as marine oils and specifically Krill oils are highly prone to oxidation to lipid peroxides and other secondary oxidation products. Oxidized oils may have altered biological activity, making them ineffective or harmful. Therefore, it is very important that marine oils have sufficient internal antioxidant capacity to diminish oxidation processes caused by free radicals generated during the storage of these marine oils. Optimal lipase activity is required in order to enable proper hydrolyses and absorption of fatty acids.

Despite the importance of delivering Krill oil preparations with an optimal endogenous mineral and metal content (specifically high calcium levels and low sodium levels), low levels of impurities (such as choline, betaine, and amino acids), and low and stable TMA levels, the inventors have surprisingly found that Krill oil products currently marketed as nutraceuticals do not contain optimal levels of minerals and metals and contain high amounts of TMA.

### SUMMARY OF THE INVENTION

The present invention relates to Krill oil preparations and compositions with low and stable amounts of trimethylamine (TMA) and/or optimal mineral and metals content and/or low amounts of impurities.

In certain embodiments of the present invention, the Krill oil preparation comprises a Krill oil wherein the Krill oil has a concentration of 5 mgN/100 g or less of trimethylamine. In certain embodiments of the present invention, the Krill oil has 5 mgN/100 g or less of TMA after three months, preferably six months, of storage at 40° C. or less. In certain other embodiments of the present invention, the Krill oil has more than 700 ppm by weight of endogenous calcium and/or less than 1200 ppm by weight of sodium. In certain other embodiments of the present invention, the Krill oil has more than 500 ppm by weight of endogenous magnesium. In even other embodiments of the present invention, the Krill oil has less than 450 ppm by weight of free choline and/or less than 1000 ppm by weight of betaine and/or less than 0.3 g/100 g of total amino acids and/or less than 0.15 g/100 g of each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, and Valine.

The present invention also provides a Krill oil preparation comprising Krill oil, wherein the Krill oil has a concentration of more than 700 ppm by weight of endogenous calcium and/or less than 1200 ppm by weight of sodium. In certain embodiments of the present invention, the Krill oil includes more than 500 ppm by weight of endogenous magnesium. In certain other embodiments of the present invention, the Krill oil includes less than 450 ppm by weight of free choline, and/or less than 1000 ppm by weight of betaine and/or less than 0.3 g/100 g of total amino acids and/or less than 0.15 g/100 g of each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, and Valine.

The present invention also provides a Krill oil composition comprising a Krill oil preparation, wherein the Krill oil preparation has a concentration of more than 700 ppm by weight of endogenous calcium and/or less than 1200 ppm by weight of sodium. In certain embodiments of the present invention, the Krill oil preparation has more than 500 ppm by weight of endogenous magnesium. In certain other embodiments of the present invention, the Krill oil preparation has less than 450 ppm by weight of free choline, and/or less than 1000 ppm by weight of betaine and/or less than 0.3 g/100 g of total amino acids and/or less than 0.15 g/100 g of each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, and Valine.

The present invention provides a composition, a capsule, a nutraceutical or a dietary supplement including any of the Krill oil preparations described herein, or a pharmaceutical composition including any of the Krill oil preparations described herein and one or more pharmaceutical excipients.

The present invention provides a composition comprising a Krill oil preparation wherein the Krill oil preparation has a concentration of 5 mgN/100 g or less of trimethylamine. In certain embodiments of the present invention, the Krill oil preparation has a concentration of 5 mgN/100 g. or less of trimethylamine after three months, preferably six months, of storage of the composition at 40° C. or less.

The present invention provides a composition comprising a Krill oil preparation wherein the Krill oil preparation includes more than 700 ppm by weight of endogenous calcium and/or less than 1200 ppm by weight of sodium. In certain embodiments of the present invention, the Krill oil preparation includes more than 500 ppm by weight of endogenous magnesium. In certain other embodiments of the present invention, the Krill oil preparation includes less than 450 ppm by weight of free choline and/or less than 1000 ppm by weight of betaine and/or less than 0.3 g/100 g of total amino acids and/or less than 0.15 g/100 g of each of the following amino acids: Alanine, Arginine, Aspartic acid, Cystine, Glutamic acid, Glycine, Histidine, Isoleucine, Leucine, Lysine, Methionine, Omithine, Phenylalanine, Proline, Serine, Hydroxyproline, Threonine, Tryptophan, Tyrosine, and Valine. In certain other embodiments of the present invention, the composition does not include minerals or metals from non-Krill sources. In certain other embodiments of the present invention, the composition is a capsule, a nutraceutical, a dietary supplement or a pharmaceutical composition that includes one or more pharmaceutical excipients.

The present invention provides a composition including a non-Krill oil preparation and a Krill oil preparation, and comprising 5 mgN/100 g or less of trimethylamine. In certain embodiments of the present invention, the Krill oil preparation includes 5 mgN/100 g or less of trimethylamine after three months, preferably six months, of storage of the composition at 40° C. or less. In certain other embodiments of the present invention, the Krill oil preparation includes more than 700 ppm by weight of endogenous calcium and/or less than 1200 ppm by weight of sodium. In certain other embodiments of the present invention, the Krill oil preparation includes more than 500 ppm by weight of endogenous magnesium. In even other embodiments of the present invention, the Krill oil preparation includes less than 450 ppm by weight of free choline, and/or less than 1000 ppm by weight of betaine and/or less than 0.3 g/100 g of total amino acids and/or less than 0.15 g/100 g for each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, and Valine.

The present invention provides a process of making a Krill oil preparation, including a step of extracting Krill oil from Krill with a solvent mixture of one or more polar solvents and one or more non-polar solvents. In certain embodiments of the present invention, the process is effective to reduce sodium in the Krill oil preparation to levels that are below levels of endogenous calcium in the Krill oil preparation. In certain other embodiments of the present invention, the process is effective to reduce sodium in the Krill oil preparation to less than 1200 ppm by weight and to maintain endogenous calcium in the Krill oil preparation at more than 700 ppm by weight.

In certain embodiments of the present invention, the solvent mixture in the process comprises hexane and ethanol. In certain other embodiments of the present invention, the solvent mixture comprises hexane and ethanol in a volume ratio of about 9:1. In certain other embodiments of the present invention, the process includes one or more steps of washing the extracted Krill oil with water. And in certain other embodiments of the present invention, the extracted Krill oil is dissolved in organic solvent mixture when the at least one step of washing is conducted.

The present invention provides a Krill oil preparation made by a process comprising a step of extracting Krill oil from Krill with a solvent mixture of one or more polar solvents and one or more non-polar solvents. In certain embodiments of the present invention, the solvent mixture comprises hexane and ethanol in a volume ratio of about 9:1.

The present invention provides methods and compositions for use in methods of reducing cardiovascular disease or disorder (CVD) risk factors (for example reducing total cholesterol, LDL-cholesterol or triglycerides, or increasing the amount of HDL-cholesterol) and/or treating or preventing CVD, and/or improving a condition in a subject suffering from CVD and/or improving a condition in a subject suffering from cognitive disease or disorder, and/or treating or preventing cognitive disease or disorder, and/or treating or preventing inflammation or inflammatory disease and/or improving a condition in a subject suffering from inflammation or inflammatory disease or disorder and/or treating or preventing depression and/or improving a condition in a subject suffering from depression and/or treating or preventing premenstrual syndrome and/or improving a condition in a subject suffering from premenstrual syndrome. Said method including administering to a human in need thereof an effective amount of a Krill oil preparation wherein the Krill oil has a trimethylamine concentration of 5 mgN/100 g or less and/or a Krill oil preparation wherein the Krill oil preparation comprises 5 mgN/100 g or less of trimethylamine.

The present invention provides methods and compositions for use in methods of reducing CVD risk factors (for example reducing total cholesterol, LDL-cholesterol or triglycerides, or increasing the amount of HDL-cholesterol) and/or treating or preventing CVD, and/or improving a condition in a subject suffering from CVD and/or improving a condition in a subject suffering from cognitive disease or disorder, and/or treating or preventing cognitive disease or disorder, and/or treating or preventing inflammation or inflammatory disease and/or improving a condition in a subject suffering from inflammation or inflammatory disease or disorder and/or treating or preventing depression and/or improving a condition in a subject suffering from depression and/or treating or preventing premenstrual syndrome and/or improving a condition in a subject suffering from premenstrual syndrome. Said method including administering to a human in need thereof an effective amount of a Krill oil preparation wherein the Krill oil has a concentration of more than 700 ppm by weight of endogenous calcium and/or less than 1200 ppm by weight of sodium, and/or an effective amount of a Krill oil preparation wherein the Krill oil preparation has a concentration of more than 700 ppm by weight of endogenous calcium and/or less than 1200 ppm by weight of sodium.

In certain embodiments of the present invention, the Krill oil preparation is made by a process comprising a step of extracting Krill oil from Krill with a solvent mixture of one or more polar solvents and one or more non-polar solvents.

### BRIEF DESCRIPTION OF THE FIGURES

FIG. 1 is a graph showing the DPPH Absorbance at 517 nm of the Krill oil preparation according to the present invention (produced according to Example 3B) and control krill oil (Brand C).

### DETAILED DESCRIPTION OF THE INVENTION

The present invention discloses for the first time Krill oil and preparations thereof with optimal levels of minerals and metals, low levels of impurities and low and stable levels of TMA.

The terms “endogenous calcium levels” or “endogenous magnesium levels” refer to calcium or magnesium levels which are extracted from the Krill biomass without the addition of natural or synthetic calcium or magnesium.

In one embodiment of the present invention, the Krill oil contains high endogenous calcium levels, preferably ≥700 ppm, low levels of sodium, preferably ≤1200 ppm and/or low TMA levels, preferably ≤5 mgN/100 g, wherein the TMA levels remain stable at ≤5 mgN/100 g during storage at room temperature over a period of one month.

According to another embodiment of the present invention, the endogenous calcium level in the Krill oil preparation is above 700 ppm, preferably above 1000 ppm, more preferably above 1200 ppm, even more preferably above 2000 and most preferably above 3000 or 4000 ppm.

According to another embodiment of the present invention, the sodium level in the Krill oil preparation is below 1200 ppm, preferably below 1100 ppm, more preferably below 1000, even more preferably below 900 ppm and most preferably below 700 or 500 ppm.

According to another embodiment of the present invention, the levels of calcium in the Krill oil preparation are higher than the levels of sodium, preferably the ratio of Ca/Na is >1, more preferably >2, even more preferably >3 and most preferably >4.

In one embodiment of the present invention, the TMA level of the Krill oil preparation does not increase above 5 mgN/100 g, preferably 4 mgN/100 g, more preferably 3 mgN/100 g and most preferably 1 mgN/100 g, after storage for at least 4 months. In another embodiment of the present invention, the TMA level does not increase above 5 mgN/100 g, preferably 4 mgN/100 g, more preferably 3 mgN/100 g and most preferably 1 mgN/100 g, after storage for at least 5 months, for at least 6 months, for at least 7 months, for at least 8 months, for at least 9 months, for at least 10 months, for at least 11 months, or for at least one year.

In one preferred embodiment of the present invention, the TMA level of the Krill oil preparation does not increase above 5 mgN/100 g, preferably 4 mgN/100 g, more preferably 3 mgN/100 g and most preferably 1 mgN/100 g, during a period of at least 6 months at ambient temperature (20-30° C.), or during a period of at least 3 months at 40° C. or less.

In one embodiment of the present invention, the Krill oil preparation contains high endogenous magnesium levels of above 500 ppm, preferably above 750 ppm, more preferably above 1000 ppm and most preferably above 2000 ppm.

In one embodiment of the present invention, the Krill oil preparation contains low free choline levels, preferably less than 450 ppm, more preferably less than 300 ppm even more preferably less than 200 ppm and most preferably less than 100 ppm.

In one embodiment of the present invention, the Krill oil preparation contains low betaine levels, preferably less than 1000 ppm, more preferably less than 750 or 500 ppm, even more preferably less than 250 ppm and most preferably less than 50 ppm or less than 10 ppm.

In one embodiment of the present invention, the Krill oil preparation contains low total amino acids levels, preferably less than 0.3 g/100 g, more preferably less than 0.1 g/100 g and most preferably less than 0.05 g/100 g.

According to another embodiment of the present invention, the Krill oil preparation contains low levels of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, Valine. Preferably, the levels of each of one of said amino acids is less than 0.15 g/100 g or 0.1 g/100 g, preferably less than 0.05 g/100 g, more preferably less than 0.04 g/100 g, even more preferably less than 0.02 g/100 g and most preferably less than 0.006 g/100 g.

According to another embodiment of the present invention, the Krill oil preparation comprises at least 2 g/100 g phospholipids, preferably above 10 g/100 g, more preferably above 25 g/100 g, and most preferably above 35 g/100 g or 40 g/100 g phospholipids.

According to another embodiment of the present invention, the Krill oil preparation comprises at least 3% EPA, preferably above 5% EPA, more preferably above 6% EPA and most preferably above 8% or 11% EPA. According to another embodiment of the present invention, the Krill oil preparation comprises at least 2% DHA, preferably above 3% DHA and more preferably above 5% or 9% DHA.

The Krill oil preparation of the present invention may be in the form of fluid oil, powder, granules, wax, paste, oil or aqueous emulsion, and any other form that will enable its use. In a further aspect of the present invention, the Krill oil preparation is used in conjunction with or is part of a nutritional, pharmaceutical or nutraceutical composition or a functional or medical food.

A nutritional composition as used herein can be any nutritional composition including, but not limited to, human milk fat substitute, infant formula, dairy product, milk powder, drinks, ice-cream, biscuit, soy product, bakery, pastry and bread, sauce, soup, prepared food, frozen food, condiment, confectionary, oils and fat, margarine, spread, filling, cereal, instant product, infant food, toddler food, bar, snack, candy and chocolate product.

A functional food as used herein can be any functional food, including, but not limited to, dairy product, ice-cream, biscuit, soy product, bakery, pastry, cakes and bread, instant product, sauce, soup, prepared food, frozen food, condiment, confectionary, oils and fat, margarine, spread, filling, cereal, instant product, drinks and shake, infant food, bar, snack, candy and chocolate product.

A nutraceutical composition as used herein can be any nutraceutical, which can be any substance that may be considered as a food or part of a food and provides medical or health benefits, including the prevention and treatment of diseases or disorders. Such nutraceutical compositions include, but are not limited to, a food additive, a food supplement, a dietary supplement, genetically engineered foods such as for example vegetables, herbal products, and processed foods such as cereals, soups and beverages and stimulant functional food, medical food and pharmafood. Dietary supplements may be delivered in the form of soft gel capsules, tablets, syrups, and other known dietary supplement delivery systems.

The pharmaceutical or nutraceutical compositions may be in any of the many dosage delivery forms commonly used in the art. Pharmaceutical compositions suitable for oral administration may be presented as discrete dosage units such as pills, tablets, pellets, dragées, or capsules, or as a powder or granules, or as a solution, suspension or elixir.

Suitable routes of administration for the compositions of the subject invention are oral, buccal, sublingual administration, administration via a feeding tube, topical, transdermal, or parenteral (including subcutaneous, intramuscular, intravenous and intradermal) administration. In one embodiment, the compounds are administered orally.

The present invention also provides pharmaceutical compositions wherein the Krill oil preparation is admixed with (pharmaceutically) acceptable auxiliaries, and optionally other therapeutic agents. The auxiliaries must be “acceptable” in the sense of being compatible with the other ingredients of the composition and not deleterious to the recipients thereof.

In one embodiment of the present invention, a pharmaceutical composition of the present invention further comprises at least one additional pharmaceutically active agent.

The present invention provides a method of reducing CVD risk factors (for example reducing total cholesterol, LDL-cholesterol or triglycerides, or increasing the amount of HDL-cholesterol) and/or treating or preventing CVD, and/or improving a condition in a subject suffering from CVD and/or improving a condition in a subject suffering from cognitive disease or disorder, and/or treating or preventing cognitive disease or disorder, and/or treating or preventing inflammation or inflammatory disease and/or improving a condition in a subject suffering from inflammation or inflammatory disease or disorder and/or treating or preventing depression and/or improving a condition in a subject suffering from depression and/or treating or preventing premenstrual syndrome and/or improving a condition in a subject suffering from premenstrual syndrome. Said method including administering to a human in need thereof an effective amount of a Krill oil preparation wherein the Krill oil preparation comprises 5 mgN/100 g or less of trimethylamine.

The present invention provides a method of reducing CVD risk factors (for example reducing total cholesterol, LDL-cholesterol or triglycerides, or increasing the amount of HDL-cholesterol) and/or treating or preventing CVD, and/or improving a condition in a subject suffering from CVD and/or improving a condition in a subject suffering from cognitive disease or disorder, and/or treating or preventing cognitive disease or disorder, and/or treating or preventing inflammation or inflammatory disease and/or improving a condition in a subject suffering from inflammation or inflammatory disease or disorder and/or treating or preventing depression and/or improving a condition in a subject suffering from depression and/or treating or preventing premenstrual syndrome and/or improving a condition in a subject suffering from premenstrual syndrome. Said method including administering to a human in need thereof an effective amount of a Krill oil preparation wherein said Krill oil preparation comprises more than 700 ppm by weight of endogenous calcium and less than 1200 ppm by weight of sodium.

The term “CVD risk factors” as used herein should be understood to encompass, among others, high blood LDL or total cholesterol and triglyceride levels, low serum HDL cholesterol, elevated serum homocysteine, high blood pressure, inflammation, diabetes and overweight and obesity (NHLBI. (2011) What Are Coronary Heart Disease Risk Factors?).

The term “CVD” as used herein should be understood to encompass any cardiovascular disease or disorder. Non-limiting examples of such a cardiovascular disease or disorder include rheumatic heart disease, heart valve disease, aneurysm, atherosclerosis, peripheral arterial disease, angina, coronary artery disease, coronary heart disease, myocardial infarction, sudden death, cerebrovascular disease, stroke, transient ischemic attacks, cardiomyopathy, pericardial disease, congenital heart disease and heart failure.

The term “cognitive disease or disorder” as used herein should be understood to encompass any cognitive disease or disorder. Non-limiting examples of such a cognitive disease or disorder are Attention Deficit Disorder (ADD), Attention Deficit Hyperactivity Disorder (ADHD), dyslexia, age-associated memory impairment and learning disorders, amnesia, mild cognitive impairment, cognitively impaired non-demented, pre-Alzheimer's disease, Alzheimer's disease, Parkinson's disease, pre-dementia syndrome, dementia, age related cognitive decline, cognitive deterioration, moderate mental impairment, mental deterioration as a result of aging, conditions that influence the intensity of brain waves and/or brain glucose utilization, stress, anxiety, depression, behavior disorders, concentration and attention impairment, mood deterioration, general cognitive and mental well-being, neurodegenerative disorders, hormonal disorders or any combinations thereof. In a specific embodiment, the cognitive disorder is memory impairment.

The term “inflammatory disease” as used herein should be understood to encompass any inflammatory disease or disorder. Non-limiting examples of such an inflammatory disease or disorder include rheumatoid arthritis, osteoarthritis, asthma, prostatitis, colitis, Crohn's disease, dermatitis, diverticulitis, glomerulonephritis, interstitial cystitis, irritable bowel syndrome, nephritis, pelvic inflammatory disease, periodontitis, reperfusion injury, sarcoidosis, transplant rejection and vasculitis.

The term “improving a condition” as used herein should be understood to encompass: ameliorating undesired symptoms associated with a disease, disorder, or pathological condition; preventing manifestation of symptoms before they occur; slowing down progression of a disease or disorder; slowing down deterioration of a disease or disorder; slowing down irreversible damage caused in a progressive (or chronic) stage of a disease or disorder; delaying onset of a (progressive) disease or disorder; reducing severity of a disease or disorder; curing a disease or disorder; preventing a disease or disorder from occurring altogether (for example in an individual generally prone to the disease) or a combination of any of the above.

The effective amount of the preparation claimed herein is the dose of this preparation that provides a therapeutic benefit in the treatment or management of the disclosed conditions and diseases. A person skilled in the art would recognize that the effective amount may vary, for example, depending on known factors such as the pharmacodynamic and pharmacokinetic characteristics of the inventive preparation and its mode and route of administration; the age, sex, health and weight of the subject receiving the preparation; the frequency of the treatment and the effect desired; and the kind of the concurrent treatment. A person skilled in the art would also recognize that the effective amount, or dose, of the preparation can be determined based on the disclosures in this patent application and common knowledge in the art.

The amount of the preparation that will be effective in the treatment and/or management of the conditions and diseases disclosed herein can be determined by standard clinical techniques. In vitro or in vivo assays may optionally be employed to help identify optimal dosage ranges. The present invention also provides a process of making a Krill oil preparation. Previous art describing production of marine oil from marine biomass focuses mainly on optimizing the yield and the lipid composition of the oil (PCT Publications WO 00/23546 (Beaudoin), WO 00/23546 (extraction according to Folch et al., J Biol Chem. 1957; 226:497-509), Yamaguchi et al., 1986). Surprisingly, the inventors of the present invention have found that non lipid substances, which have critical effects on nutritional, stability and safety aspects of the oil, can be controlled by the process of manufacturing the oil preparation. It was surprisingly found that the method by which Krill oil is extracted from Krill significantly influences the amount of minerals, metals and impurities (such as choline, betaine, and amino acids) in the resulting Krill oil preparation.

It was also surprising to find out that the specific mineral and metal profile can be manipulated by the extraction process and, optionally, by the subsequent washing process.

Furthermore, the inventors surprisingly found that the process of extracting Krill oil from Krill influences the oil's TMA level over time. As demonstrated in Example 5, conventional extraction methods result in Krill oil preparations which contain TMA levels that increase over time, and an undesirable mineral and metal composition.

These unique findings enabled the inventors to form a novel composition of Krill oil that contains optimal mineral and metal composition, low impurities (such as choline, betaine, and amino acids) and also low levels of TMA which remain low for long periods.

According to one embodiment of the present invention, the Krill oil preparation has improved anti-oxidant capacity, improved lipase activity and improved transepithelial calcium transport, by comparison to Krill oil preparations known in the art (which contain<700 ppm calcium, >1200 ppm sodium and/or >5 mgN/100 g TMA).

The present invention provides a process of preparing the Krill oil preparations of the invention, comprising extraction process and optional washes.

The stage of extracting the oil is optionally and preferably performed by adding one or more organic solvents to the Krill biomass to form the oil extract. The Krill biomass may be in the form of meal or in the form of fresh or frozen Krill, or in the form of fresh or frozen krill that was processed by cooking and decantation to remove some of the water content or in any other krill form. Optionally and more preferably, the liquid phase (containing the oil dissolved in the organic solvent) is separated from the defatted biomass by centrifugation, filtration, gravity separation or other means. Optionally, residual krill oil left with the defatted biomass is extracted from the biomass by repeating of the process described above: addition of one or more organic solvents to the defatted krill biomass and separation of the liquid by the same optional means (i.e. centrifugation, filtration, gravity separation etc.). The liquid phases obtained from extraction and re-extractions are united to form final liquid phase.

In case filtration is selected as the separation method, the repeated extraction may preferably be performed by simply washing the defatted biomass left as a “filtration cake” after first liquid phase was removed from it. This re-extraction done by washing will be performed, again, by using one or more organic solvent. The filtrates, i.e. the liquid phases, obtained from extraction and re-extractions are united to form final liquid phase.

The final liquid phase is optionally washed by adding water, optionally also adding an organic solvent, mixing the water and the organic solvent with the final liquid phase. After mixing, separation is performed optionally by gravity or by centrifugation forming two distinct phases: organic phase containing the krill oil and a second phase containing most of the water (i.e. water phase). The organic phase can be optionally washed again with water and optionally an organic solvent in the same procedure. The final liquid phase, whether washed or not, is optionally and preferably subjected to evaporation stage in order to remove the organic solvent and obtain oil. Evaporation is preferably done under reduced pressure.

The ratio between solvent and Krill biomass (V solvent:W biomass) during the extraction or re-extraction stages is less than 10:1, preferably less than 5:1, more preferably less than 4:1.

Extraction conditions should be controlled and can, optionally, be maintained between 10-60° C., preferably between 30-40° C., and for 1 minute to 10 hours, preferably for 1-3 hours, and more preferably for 2-2.5 hours. The extraction may be done batchwise, for example in a batch reactor, or optionally by a continuous extraction process. Continuous extraction can be done in co-current or counter current mode in continues extraction systems such as those known in the art. The ratio between solvent to krill biomass in continuous extraction is considered as the ratio between the flow rates of the two streams in the system.

The water washes stage may optionally be conducted continuously as well. Optionally, water and organic phases may be mixed by in-line mixer or by CSTR or by mixers-settlers systems The mixed water-organic phases may be passed through continues or batch gravity separation tanks or, optionally, be separated by continues centrifugation. In case of continuous washing, the ratios between water, ethanol and organic phase will be considered as the ratio between the flow rate of each of those streams.

The organic solvent preferably, but not limited, comprises organic solvents which comprise, optionally, a mixture of polar and non-polar solvents. Polar solvent may include: ethanol, methanol, 2-propanol, butanol and such. Non polar solvent may be from the group of one or more of the following: hexane, heptane, petroleum ether and others. The ratio between polar and non-polar solvents (volume:volume) is preferably 1:99-99:1, more preferably 5:95-50:50, more preferably 10:90-20:80. Preferred solvent mixture is hexane ethanol mixture.

The volume of the water phase that is used for washing the organic phase (containing oil dissolved in organic solvents) during the washes stage is optionally less than 100% of the final liquid phase volume, preferably less than 50% of the final liquid phase, more preferably less than 10% of the final liquid phase volume.

The oil obtained following evaporation is optionally and preferably subjected to a water washes stage in which the oil is preferably re-dissolved in an organic solvent to form an organic phase. Water is added, optionally together with the organic solvents, or optionally after them, to the organic phase, mixed together with it and separated from it by gravity separation or by centrifugation. The water wash stage can optionally be repeated once or several times. Final krill oil preparation will be obtained by removing the solvents from the washed oil, optionally by evaporation of the organic solvent, preferably under reduced pressure.

The ratio between oil and organic solvent that are forming the organic phase when conducting the water washes is preferably (oil:organic solvent w/v) 1:1-1:40, preferably 1:2-1:30, more preferably 1:3-1:10 and most preferably 1:5-1:8.

The organic solvent preferably, but not limited, comprises organic solvents which comprise, optionally, a mixture of polar and non-polar solvents. Polar solvent may include: ethanol, methanol, butanol and such. Non polar solvent may be from the group of one or more of the following: hexane, heptane and others. The ratio between polar and non-polar solvents (V:V) is preferably 1:99-99:1, more preferably 5:95:50:50, more preferably 10:90-20:80. Preferred solvent mixture is hexane ethanol mixture.

The volume of the water phase that is used for washing the organic phase (containing oil+organic solvent) during the washes stage is preferably less than 100% of the organic phase volume, preferably less than 50% of the organic phase, more preferably less than 40% of the organic phase, even more preferably less than 30% of the organic phase and most preferably less than 10% of the organic phase volume.

TMA levels in krill oil samples were tested by an external laboratory, Nofima BioLab, Norway. Measurements were conducted in Conway dishes according to a modified version of Conway and Byrne's micro-diffusion method (Conway & O'Malley, Microdiffusion Methods. Biochem, 36, 656-661 (1942)).

Phospholipids (PL) content in krill oil samples was analyzed by P-NMR by 3rd party lab (Spectral Services) or calculated from HPTLC analysis. HPTLC analysis was performed by dissolving the sample with chloroform:methanol 95:5 v/v solution, and running it on HPTLC silica gel plate using an eluent solution including water, methanol, acetic acid, acetone and chloroform, and staining the plate with a staining solution containing water, sulfuric acid and anhydrous copper sulfate. EPA and DHA contents were analyzed by gas chromatography—modified AOCS official method Ce 1b-89.

Elemental analysis to determine mineral and metal content in Krill oil samples was performed by ICP method by POS Bio-sciences, Canada.

Choline and Betaine in krill oil samples were analyzed by Eurofins Analytik GmbH, Germany using LC-MS-MS. Total amino acids content was also analyzed by Eurofins Analytik GmbH, according to reference method ISO 13903:2005; EU 152/2009 (F) and ISO 13904:2005; EU 152/2009 (F).

The accelerated stability test is a standard accelerated storage conditions model for drug substances and products (“Stability Testing of New Drug Substances and Products Q1A(R2)”, ICH Harmonised Tripartite Guideline, February 2003).

The terms parts per million (ppm) and percent (%) as used herein in connection with amounts and concentrations of compounds mean parts per million by weight and weight percent, respectively.

### EXAMPLES

### Example 1: Mineral and Metal Composition and Content of Choline, Betaine and Amino Acids of Marketed Krill Oil Capsules

Capsules of commercially available Krill oil products, purchased from consumer sources, were tested for metals and minerals composition (Table 1). It can be seen that the values of sodium in the tested capsules are above 1200 ppm and the values of calcium are below 700 ppm. In both cases, the sodium levels are higher than the calcium levels. It can also be seen that the choline levels are above 450 ppm, the betaine levels are above 1000 ppm and the amino acids levels are above those obtained in the Krill oil of the invention.

TABLE 1
Metals, minerals and amino acids composition
of marketed Krill oil capsules.

| Brand | C | L |
|---|---|---|
| Mineral composition: | | |
| Aluminum (μg/g) | 3.6 | <0.5 |
| Barium (μg/g) | 0.18 | <0.05 |
| Boron (μg/g) | <1 | <1 |
| Calcium (μg/g) | 470 | 320 |
| Copper (μg/g) | 0.26 | 2.5 |
| Iron (μg/g) | 1 | <0.5 |
| Magnesium (μg/g) | 120 | 720 |
| Manganese (μg/g) | 0.2 | <0.1 |
| Potassium (μg/g) | 1100 | 280 |
| Sodium (μg/g) | 2600 | 1300 |
| Sulfur (μg/g) | 650 | 420 |
| Zinc (μg/g) | 3.7 | 1.5 |
| Betaine (μg/g) | NT | 1080 |
| Choline (free) (μg/g) | NT | 478 |
| Amino acids composition: | | |
| Alanine (g/100 g) | NT | 0.018 |
| Arginine (g/100 g) | NT | 0.047 |
| Aspartic acid (g/100 g) | NT | <0.017 |
| Cysteine + Cystine (g/100 g) | NT | <0.006 |
| Glutamic acid (g/100 g) | NT | <0.021 |
| Glycine (g/100 g) | NT | 0.024 |
| Histidine (g/100 g) | NT | <0.02 |
| Isoleucine (g/100 g) | NT | <0.035 |
| Leucine (g/100 g) | NT | <0.015 |
| Lysine (g/100 g) | NT | <0.014 |
| Methionine (g/100 g) | NT | <0.024 |
| Phenylalanine (g/100 g) | NT | 0.058 |
| Proline (g/100 g) | NT | 0.168 |
| Serine (g/100 g) | NT | <0.016 |
| Threonine (g/100 g) | NT | <0.006 |
| Tryptophan (g/100 g) | NT | <0.01 |
| Tyrosine (g/100 g) | NT | <0.023 |
| Valine (g/100 g) | NT | <0.016 |

NT = not tested

### Example 2: TMA Levels of Marketed Krill Oil Capsules

Capsules of commercially available products, purchased from consumer sources, were tested for TMA levels. Surprisingly, TMA levels were found to be higher than 5 mgN/100 g when sampled (Table 2). Thus, the tested Krill oil had high TMA levels, although the tests were conducted within the recommended shelf life of the product (as declared in the expiry date).

TABLE 2
TMA composition of marketed Krill oil capsules

| | | | TMA |
|---|---|---|---|
| Brand | Expiry date | Analysis Date | (mgN/100 g) |
| A | March 2014 | June 2013 | 9 |
| B | August 2014 | January 2013 | 15 |
| C | December 2014 | January 2013 | 15 |
| D | September 2013 | January 2013 | 14 |
| E | October 2015 | January 2013 | 13 * |
| F | February 2012 | October 2010 | 34 |
| G | February 2012 | October 2010 | 12 |
| H | March 2011 | October 2010 | 7 |
| I | December 2012 | October 2010 | 10 |
| J | October 2013 | October 2010 | 17 |
| K | December 2012 | October 2011 | 8 |

* TMA levels tested after a stability test, involving storage of the capsules at 40° C. for one month, increased from 13 mgN/100 g to 28 mgN/100 g.

### Example 3: Production of Krill Oil According to the Present Invention

A. Extraction of oil from Krill meal was performed by addition of 4333 l solvents to 1300 kg Krill meal in a batch reactor and mixing at about 40° C. for about 2 hours. The mixture of solvents contained hexane and ethanol in a volume ratio of 90:10 respectively. The filtration of the solvents including the extracted oil was performed using a basket centrifuge system after reactor was cooled down to about 25° C. The defatted meal powder was discharged from the basket centrifuge and re-extracted with additional 2520 l of the same mixture of solvents, in the same batch reactor, and the resulting solvent/oil mixture was then filtered again in a basket centrifuge. All filtrates were united.

The oil was washed in a mixture containing hexane ethanol and water. After clear phase separation was obtained, the bottom (water) phase was removed. The solvents of the organic (top) phase were evaporated under reduced pressure at about 40-50° C. for about 12 hours to produce Krill oil.

The described process resulted in about 250 kg Krill oil containing: PL (phospholipids)=42.12 g/100 g; EPA=11.4 g/100 g; DHA=9.2 g/100 g; TMA measurements showed TMA<1 mgN/100 g. After a stability test of 8 months at room temperature TMA levels remained<1 mgN/100 g. Elemental analysis results of the produced oil are presented in Table 3.

B. Extraction of oil from Krill meal was performed by addition of 1200 ml solvent to 300 gr Krill meal and shaking at about 40° C. for about 2 hours. Solvents mixture contained hexane and ethanol in a volume ratio of about 90:10 respectively. Filtration of the solvents, including extracted oil, from the meal powder was performed using a Buchner vacuum system. The defatted meal powder, left as the “filtration cake”, was washed with additional 600 ml of the same solvent mixture in order to further extract oil left in the defatted meal. All filtrates were united, and the solvents were evaporated under reduced pressure in rotary evaporator, with a bath at about 50° C. for about 1 hour until less than 10 mbar vacuum was achieved and there was no visible boiling in the oily phase. About 65 g of oil were obtained.

About 30 g out of the 65 g of the obtained oil were dissolved in about 206 ml solvent mixture including hexane, ethanol and water. The solution was stirred and the organic and water phases were allowed to separate in a separation funnel. The solvents of the organic (top) phase were evaporated to produce Krill oil under reduced pressure in a rotary evaporator, with a bath at about 50° C. for about 1 hour until less than 10 mbar vacuum was achieved and there was no visible boiling in the oily phase.

The described process resulted in krill oil preparation containing: PL>25 g/100 g; EPA>8 g/100 g; DHA>4.5 g/100 g. Elemental analysis results of the produced oil are presented in Table 3.

TABLE 3
Metals and minerals composition of Krill
oil produced in Examples 3A and 3B.

| Elemental Analysis | Results for oil | Results for oil |
|---|---|---|
| Units: ug/g | of example 3A | of example 3B |
| Aluminum | 1.8 | 1.1 |
| Barium | 0.09 | 1.2 |
| Boron | <1 | 1 |
| Calcium | 3100 | 4600 |
| Copper | 2.3 | 4.6 |
| Iron | 0.9 | 3.1 |
| Magnesium | 1200 | 2500 |
| Manganese | 0.2 | 0.5 |
| Potassium | 370 | 420 |
| Sodium | 1100 | 860 |
| Sulfur | 820 | 880 |
| Zinc | 2.6 | 2.5 |

C. Krill oil was prepared according to the present invention, in a method equivalent to method B to the examples described above. The process resulted in krill oil preparation containing: PL=40.95 g/100 g; EPA=12.1 g/100 g; DHA=6.7 g/100 g; free choline=323 ppm; betaine<2 ppm and the following amino acids composition:

| Amino acid | g/100 g |
|---|---|
| Alanine | <0.015 |
| Arginine | <0.042 |
| Aspartic acid | <0.017 |
| Cysteine + Cystine | <0.006 |
| Glutamic acid | <0.021 |
| Glycine | <0.019 |
| Histidine | <0.02 |
| Isoleucine | <0.035 |
| Leucine | <0.015 |
| Lysine | <0.014 |
| Methionine | <0.024 |
| Phenylalanine | 0.033 |
| Proline | <0.02 |
| Serine | <0.016 |
| Threonine | <0.006 |
| Tryptophan | <0.01 |
| Tyrosine | <0.023 |
| Valine | <0.016 |

D. Extraction of oil from Krill meal was performed by addition of 800 ml solvent to 200 gr Krill meal and shaking at about 40° C. for about 2 hours. Solvents mixture contained hexane and ethanol in a volume ratio of about 90:10 respectively. Filtration of the solvents, including extracted oil, from the meal powder was performed using a Buchner vacuum system. The defatted meal powder, left as the “filtration cake”, was washed with additional 400 ml of the same solvent mixture in order to further extract oil left in the defatted meal. All filtrates were united, and the solvents were evaporated under reduced pressure in rotary evaporator, with a bath at about 50° C. for about 1 hour until less than 10 mbar vacuum was achieved and there was no visible boiling in the oily phase. About 50 g of oil were obtained.

About 30 g out of the 50 g of the obtained oil were dissolved in about 930 ml solvent mixture including hexane, ethanol and water in the following volume ratio: 87.1% hexane, 9.7% ethanol, 3.2% water. The solution was stirred and the organic and water phases were allowed to separate in a separation funnel. The solvents of the organic (top) phase were evaporated to produce Krill oil under reduced pressure in a rotary evaporator, with a bath at about 50° C. for about 1 hour until less than 10 mbar vacuum was achieved and there was no visible boiling in the oily phase.

The described process resulted in krill oil preparation containing: PL=25.8 g/100 g; EPA=8.2 g/100 g; DHA=4.8 g/100 g. TMA measurements showed TMA<1 mgN/100 g. After a stability test of 8 months at 40° C. TMA levels remained<1 mgN/100 g.

E. Krill oil was extracted using continuous industrial unit in counter current flow. The extraction was performed at about 40° C. with solvents mixture containing hexane and ethanol in a volume ratio of about 90:10 respectively. System parameters were set to ensure a flow of 300 kg/hour krill meal and 1140 L/hour solvent. The solvents containing the dissolved oil were separated continuously by gravity from the defatted meal. The solvents were evaporated under reduced pressure at about 50° C. 400 kg of the received oil was dissolved in about 2748 l solvent mixture including hexane, ethanol and water. The solution was stirred and the organic and water phases were allowed to separate. The solvents of the organic (top) phase were evaporated to produce Krill oil under reduced pressure.

The obtained oil was subjected to a second wash with same solvent mixture composed of hexane, ethanol and water.

The described process resulted in Krill oil preparation containing: PL=36.4 g/100 g; EPA=11.2 g/100 g; DHA=6.5 g/100 g; TMA<1 mgN/100 g; free choline=87.1 ppm; betaine<2 ppm; Ca=1800 ppm; Na=400 ppm and the following amino acid composition:

| Amino acid | g/100 g |
|---|---|
| Alanine | <0.015 |
| Arginine | <0.042 |
| Aspartic acid | <0.017 |
| Cysteine | <0.006 |
| Glutamic acid | <0.021 |
| Glycine | <0.019 |
| Histidine | <0.02 |
| Hydroxyproline | <0.05 |
| Isoleucine | <0.035 |
| Leucine | <0.015 |
| Lysine | <0.014 |
| Methionine | <0.024 |
| Ornithine | <0.05 |
| Phenylalanine | <0.031 |
| Proline | <0.02 |
| Serine | <0.016 |
| Threonine | <0.006 |
| Tryptophan | <0.01 |
| Tyrosine | <0.023 |
| Valine | <0.016 |

### Example 4: Production of Krill Oil Capsules According to the Present Invention

The stability of TMA low levels is demonstrated on krill oil after soft gel encapsulation, and not only as bulk oil. Krill oil is prepared according to the present invention, in an equivalent process to the examples described above and encapsulated in 333 mg soft gel capsules. Main shell ingredients used are gelatin, glycerine and water. Capsules are dried in a dry air room at ambient conditions.

TMA measurements of the krill oil in the capsules show TMA<1 mgN/100 g. After stability test of 5 months at 40° C., TMA levels remain lower than 5 mgN/100 g.

### Example 5: Production of Krill Oil According to Methods Known in the Art

In order to demonstrate the influence of extraction procedure on the final oil composition, we performed two different conventional extraction procedures based on known commercial extraction processes (Krill oil GRAS notification (GRN000371) (A) and PCT WO 00/23546 (Beaudoin) (B)). As demonstrated below, the conventional extraction processes resulted in Krill oil compositions different from the compositions of the present invention.

A. Extraction of oil from Krill meal was performed by addition of ethanol to Krill meal and shaking at 40° C. for 2 hours. The extraction, filtration and evaporation procedures were done in a way equivalent to the protocols specified in the Examples given above. Elemental analysis results of the produced oil are presented in Table 4.

B. Extraction of oil from Krill meal was performed by addition of ethanol to Krill meal and shaking at 40° C. for 2 hours. The extraction, filtration and evaporation procedures were done in a way equivalent to the protocols specified in the Examples given above. TMA measurements showed TMA=21 mgN/100 g after preparation.

C. Extraction of oil from frozen raw Krill sample was performed by addition of acetone to frozen Krill. The mixture was put in a shaker kept at 4° C. for about 2 hours. The extraction, filtration and evaporation procedures were done in an equivalent way to the protocols specified in the examples given above. TMA measurements showed TMA=<1 mgN/100 g, however, after stability test of 6 months at room temperature, TMA levels surprisingly raised to 9 mgN/100 g. After stability testing at 40° C. for 6 months TMA levels surprisingly raised to 56 mgN/100 g Elemental analysis results of the produced oil are presented in Table 4.

TABLE 4
Metals and minerals composition of Krill
oil produced in Examples 5A and 5C.

| Elemental Analysis | Results for oil | Results for oil |
|---|---|---|
| Units: ug/g | of example 5A | of example 5C |
| Aluminum | <0.5 | NT |
| Barium | 0.53 | NT |
| Boron | 1 | NT |
| Calcium | 490 | 19.8 |
| Copper | 29 | 0.19 |
| Iron | 2 | NT |
| Magnesium | 630 | 27.9 |
| Manganese | 0.2 | NT |
| Potassium | 5200 | 1980 |
| Sodium | 18300 | 6760 |
| Sulfur | 1300 | 7650 |
| Zinc | 2.1 | 1.59 |

NT = not tested

### Example 6: Evaluation of Krill Oil's Free Radical Scavenging Capacity by DPPH Assay

DPPH (2,2-diphenyl-1-picrylhydrazyl) is a well-known radical and a trap (“scavenger”) for other radicals. Therefore, rate reduction of a chemical reaction upon addition of DPPH is used as an indicator of the radical nature of that reaction. Because of a strong absorption band centered at about 517 nm, the DPPH radical has a deep violet color in solution, and it becomes colorless or pale yellow when neutralized. This property allows visual monitoring of the reaction, and the number of initial radicals can be counted from the change in the optical absorption at 517 nm.

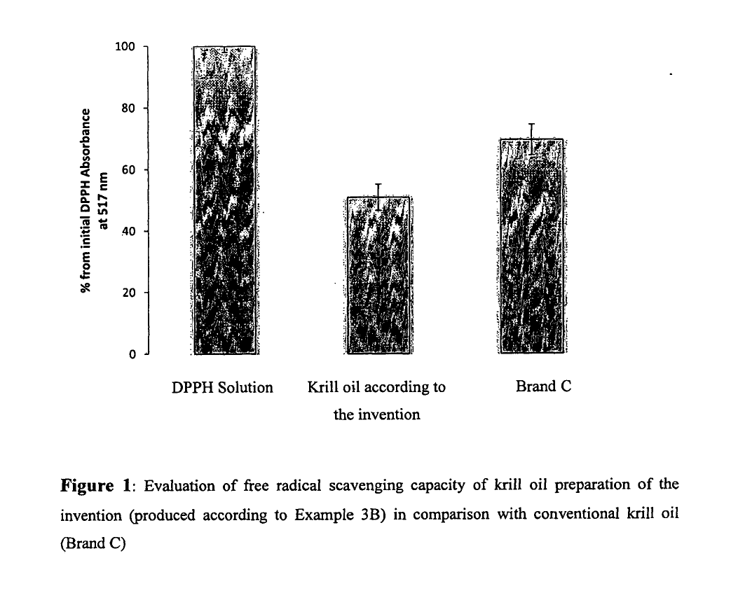

Five milliliters of 0.10 mM DPPH in isooctane (Isooctane could dissolve both DPPH and krill oil samples) were mixed with 50 mg oil samples in a 20 ml bottle and after 30 min standing in the dark, the absorbance of the sample mixture was measured at 517 nm using a UV spectrophotometer (JASCO V-630). Results of free radical scavenging activity from DPPH method was expressed as % from initial OD of DPPH solution (517 nm). Initial absorbance values of DPPH in isooctane (without krill oil) represented the control group (100%)). Data from FIG. 1 shows that Krill oil preparation according to the present invention (produced according to Example 3B, and containing optimal levels of minerals and metals, low levels of impurities and low and stable levels of TMA, decreased significantly the absorbance of DPPH. This demonstrates that krill oils preparation according to the present invention contain higher free radical scavenging capacity then the control krill oil (see Brand C in Table 1).

### Example 7: Hydrolysis of Krill Oil by Lipase Activity Assay

Dissolve Krill oil in an organic/aqueous two-phase system at 25° C. for 30 min. Initiate the reaction by adding different amounts of lipase derivative and stirring at 150 rpm. Determine the concentration of free fatty acids (FFA) at various times by HPLC-GC method. Results show that Krill oil preparations according to the present invention express higher initial rate of hydrolysis (lower lag time), and also higher total FFA levels during hydrolysis assay compared with conventional Krill oil preparations throughout the assay time points.

### Example 8: Evaluation of Transepithelial Transport Using Caco-2 Cells Model

Grow Caco-2 cells (human colon adenocarcinoma cell line) in plastic culture flasks. After reaching 90% confluent, treat cells with Krill oil according to the present invention or conventional Krill oils (containing<700 ppm calcium and >1200 ppm sodium), enriched with exogenous calcium salt, in order to evaluate transepithelial calcium transport. Results show that Krill oil according to the present invention expresses higher paracellular calcium transport across fully differentiated Caco-2 cell compared with control Krill oil enriched with exogenous calcium salt.

### Example 9: Evaluation of Krill Oil's Reducing Activity by Cyclic Voltammetry Method

Cyclic voltammetry is a type of potentio-dynamic electrochemical method that can be used to effectively characterize the reducing ability of biological samples or food extracts. Cyclic voltammetry is performed by cycling the potential of a working electrode, and measuring the resulting current, such as oxidation-reduction reactions. Therefore, the reducing ability of Krill oil samples (with or without mixing with deionized water) is analyzed by the electrochemical method of cyclic voltammeter. Data shows significantly higher reducing activities of Krill oil preparations according to the present invention in comparison with conventional Krill oil preparations.

1. A Krill oil preparation comprising Krill oil, wherein the Krill oil has a trimethylamine concentration of 5 mg Nitrogen per 100 g (mg N/100 g) or less.

2. The Krill oil preparation of claim 1, wherein the Krill oil has a trimethylamine concentration of 5 mg N/100 g or less after three months of storage at 40° C. or less.

3. The Krill oil preparation of claim 1, wherein the Krill oil has a concentration of endogenous calcium greater than 700 ppm by weight and/or a concentration of sodium of less than 1200 ppm by weight.

4. The Krill oil preparation of claim 1, wherein the Krill oil has a concentration of endogenous magnesium greater than 500 ppm by weight.

5. The Krill oil preparation of claim 1, wherein the Krill oil has a concentration of free choline of less than 450 ppm by weight and/or a concentration of betaine of less than 1000 ppm by weight and/or a total amino acid concentration of less than 0.3 g/100 g.

6. The Krill oil preparation of claim 1, wherein the Krill oil has a concentration of free choline of less than 450 ppm by weight and/or a concentration of betaine of less than 1000 ppm by weight and/or a concentration of less than 0.15 g per 100 g for each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine, and Valine.

7. A Krill oil preparation comprising Krill oil, wherein the Krill oil has a concentration of endogenous calcium greater than 700 ppm by weight and/or a concentration of sodium of less than 1200 ppm by weight.

8. The Krill oil preparation of claim 7, wherein the Krill oil has a concentration of endogenous magnesium greater than 500 ppm by weight.

9. The Krill oil preparation of claim 7, wherein the Krill oil has a concentration of free choline of less than 450 ppm by weight, and/or a concentration of betaine of less than 1000 ppm by weight and/or a total amino acid concentration of less than 0.3 g/100 g.

10. The Krill oil preparation of claim 7, wherein the Krill oil has a concentration of free choline less than 450 ppm by weight, and/or a concentration of betaine of less than 1000 ppm by weight and/or a concentration of less than 0.15 g per 100 g for each of the following amino acids: Alanine, Arginine, Aspartic acid, Cystine, Glutamic acid, Glycine, Histidine, Isoleucine, Leucine, Lysine, Methionine, Ornithine, Phenylalanine, Proline, Serine, Hydroxyproline, Threonine, Tryptophan, Tyrosine and Valine.

11. A composition comprising the Krill oil preparation of claim 1.

12-25. (canceled)

26. The composition of claim 11, wherein the composition does not contain minerals or metals from non-Krill sources.

27. The composition of claim 11, wherein the composition is a capsule.

28. The composition of claim 11, wherein the composition is a nutraceutical.

29. The composition claim 11, wherein the composition is a dietary supplement.

30. The composition claim 11, wherein the composition is a pharmaceutical composition and comprises one or more pharmaceutical excipients.

31. A process of making a Krill oil preparation, comprising a step of extracting Krill oil from Krill with a solvent mixture comprising one or more polar solvents and one or more non-polar solvents.

32. The process of claim 31, wherein the process is effective to reduce endogenous sodium levels in the Krill oil preparation to levels that are below endogenous calcium levels in the Krill oil preparation.

33. The process of claim 31, wherein the process is effective to reduce endogenous sodium in the Krill oil preparation to less than 1200 ppm by weight and/or to maintain endogenous calcium in the Krill oil preparation at more than 700 ppm by weight.

34. The process of claim 31, wherein the solvent mixture comprises hexane and ethanol.

35. The process of claim 34, wherein the volume ratio of hexane to ethanol in the solvent mixture is about 9:1.

36. The process of claim 31, further comprising at least one step of washing the extracted Krill oil with water.

37. The process of claim 36, wherein the extracted Krill oil is dissolved in an organic solvent mixture when the at least one step of washing the extracted Krill oil with water is conducted.

38-39. (canceled)

40. A method of reducing a CVD risk factor, the method comprising administering to a human in need thereof an amount of the Krill oil preparation according to claim 1 effective to reduce the CVD risk factor.

41-42. (canceled)

43. A method of reducing a CVD risk factor, the method comprising administering to a human in need thereof an amount of the Krill oil preparation according to claim 7 effective to reduce the CVD risk factor.

44-46. (canceled)

47. The method according to claim 40, wherein reducing the CVD risk factor is selected from reducing an amount of total cholesterol, reducing the amount of triglycerides, and increasing the amount of HDL-cholesterol.

48. The method according to claim 40, wherein reducing the CVD risk factor is selected from reducing an amount of total cholesterol, reducing the amount of triglycerides, and increasing the amount of HDL-cholesterol.
